(19) United States
(12) Patent Application Publication
(10) Pub. No.: US 2010/0204588 A1
(43) Pub. Date: Aug. 12, 2010

(54) METHOD AND APPARATUS FOR DETECTING MEASUREMENT SITE OF BLOOD PRESSURE

(75) Inventors: Youn-ho KIM, Hwaseong-si (KR); Kun-soo SHIN, Seongnam-si (KR); Sang-kon BAE, Seongnam-si (KR); Jong-pal KIM, Seoul (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si, (KR)

(21) Appl. No.: 12/647,123

(22) Filed: Dec. 24, 2009

(30) Foreign Application Priority Data
Feb. 11, 2009 (KR) 10-2009-0011208

(51) Int. Cl. A61B 5/021

(52) U.S. Cl. 600/485

(57) ABSTRACT

An apparatus and method which detects a blood-pressure measurement site. The apparatus for detecting a site of a body to measure blood pressure includes a sensing unit for sensing pressures applied to a blood vessel of a site of the body, a calculation unit for calculating a waveform representing the sensed pressure, and a determination unit for determining whether the site is the optimal site.

### CROSS-REFERENCE TO RELATED APPLICATIONS

This application claims the benefit of Korean Patent Application No. 10-2009-0011208, filed on Feb. 11, 2009, in the Korean Intellectual Property Office, the disclosure of which is incorporated herein in its entirety by reference.

### BACKGROUND

1. Field

Provided are an apparatus and a method for detecting a blood-pressure measurement site.

2. Description of the Related Art

People have become more and more concerned about health in recent times. The number of patients in the United States of America (U.S.A.) suffering from chronic diseases as of the year 2008 is 78 million. Typical chronic diseases include diabetes, hypertension, cardiovascular diseases, lung diseases, and the like. Persistent monitoring is required for patients with these chronic diseases. Blood pressure is used as an index of a person's health condition. Apparatuses for measuring blood pressure are commonly used in medical institutions and at home. A systolic blood pressure is a pressure when an initial pulse sound is heard while an applied pressure is slowly reduced after a pressure is applied to a site where arterial blood passes, in order to stop the flow of blood. A diastolic blood pressure is a pressure when a pulse sound disappears. Digital hemadynamometers calculate blood pressure by detecting a waveform corresponding to a pressure measured while applying a pressure to a blood vessel. When blood pressure is measured, a pressure affecting an arterial blood vessel needs to be measured. Thus, a blood-pressure measurement site needs to be determined.

### SUMMARY

Provided is an apparatus and a method for detecting a blood-pressure measurement site to accurately measure blood pressure.

Provided is a computer readable recording medium on which a program for executing the method in a computer processor is recorded.

Method and apparatus for detecting measurement site of blood pressure are not limited described above, and may also include other aspects.

Additional aspects will be set forth in part in the description which follows and, in part, will be apparent from the description, or may be learned by practice of the illustrated embodiments.

Provided is an apparatus for detecting a site of a body to measure blood pressure, the apparatus including a sensing unit for sensing pressures applied to a blood vessel of a site of the body, a calculation unit for calculating a waveform representing the sensed pressures, and a determination unit for determining whether the site is an optimal site based on a shape of the waveform.

Provided is a method of detecting a site of a body to measure blood pressure, the method including sensing pressures applied to a blood vessel of a site of the body calculating a waveform representing the sensed pressures and determining whether the site is an optimal site based on a shape of the waveform.

Provided is a computer readable recording medium storing instructions which control at least one processor to perform a method of detecting a site of a body to measure blood pressure, the method including sensing pressures applied to a blood vessel of a site of the body calculating a waveform representing the sensed pressures and determining whether the site is an optimal site based on a shape of the waveform.

### BRIEF DESCRIPTION OF THE DRAWINGS

These and/or other aspects will become apparent and more readily appreciated from the following description of the embodiments, taken in conjunction with the accompanying drawings of which:

FIG. 1 is a block diagram of an apparatus for detecting a blood-pressure measurement site, according to the present invention;

FIG. 2 is a detailed block diagram of an exemplary embodiment of a calculation unit illustrated in FIG. 1;

FIG. 3 is a diagram of an exemplary embodiment of a signal process performed by a filtering unit;

FIG. 4 illustrates a radial artery located in a wrist, and an exemplary embodiment of a horizontal cross section of an optimal blood-pressure measurement site relative to the radial artery and the wrist;

FIG. 5A is a diagram of an exemplary embodiment of sensors arranged close to a radial artery of a wrist, and FIG. 5B is an exemplary embodiment of a diagram of waveforms of sensed pressures acquired by the respective sensors in FIG. 5A;

FIG. 6A is a diagram of an exemplary embodiment of sensors arranged along a radial artery of a wrist, and FIG. 6B is an exemplary embodiment of a diagram of waveforms of sensed pressures acquired by the respective sensors in FIG. 6A;

FIG. 7 is a diagram illustrating an exemplary embodiment of envelopes of sensed pressures in order to determine a blood-pressure measurement site;

FIG. 8 is a flowchart illustrating an exemplary embodiment of a method of detecting a blood-pressure measurement site by using one sensor according to the present invention;

FIG. 9 is a flowchart illustrating an exemplary embodiment of a method of detecting a blood-pressure measurement site by using two sensors according to the present invention; and

FIG. 10 is a flowchart illustrating a method of detecting a blood-pressure measurement site in which time is taken into consideration, according to the present invention.

### DETAILED DESCRIPTION

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on” or “connected to” another element or layer, the element or layer can be directly on or connected to another element or layer or intervening elements or layers. In contrast, when an element is referred to as being “directly on” or “directly connected to” another element or layer, there are no intervening elements or layers present. Like numbers refer to like elements throughout. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “above”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “upper” relative to other elements or features would then be oriented “lower” relative to the other elements or features. Thus, the exemplary term “upper” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Reference will now be made in detail to embodiments, examples of which are illustrated in the accompanying drawings. In this regard, the exemplary embodiments may have different forms and do not be construed as being limited to the descriptions set forth herein. Accordingly, the embodiments are merely described below, by referring to the figures, to explain aspects of the present invention.

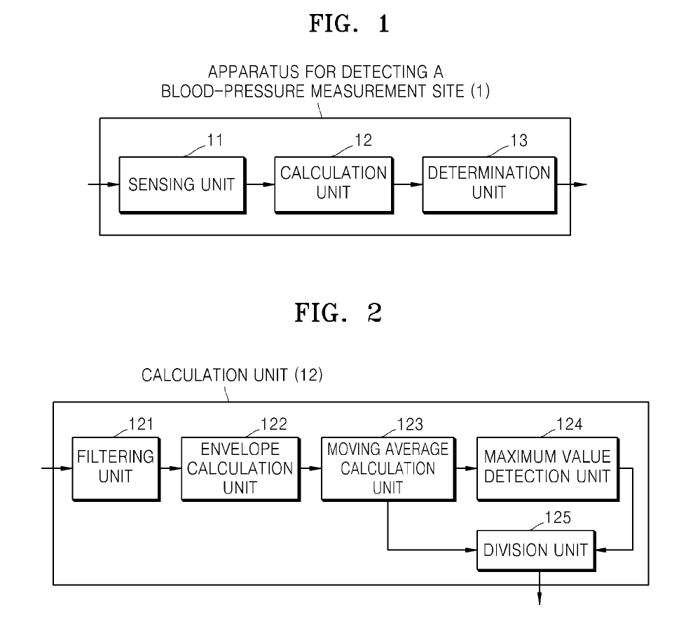

FIG. 1 is a block diagram of an exemplary embodiment of an apparatus 1 for detecting a blood-pressure measurement site, according to the present invention. Referring to FIG. 1, the apparatus 1 according to the illustrated embodiment includes a sensing unit 11, a calculation unit 12, and a determination unit 13. In general, the apparatus 1 may be included in an apparatus for measuring blood pressure, such as a blood pressure instrument, blood pressure meter, a blood pressure measurement device, or a hemadynamometer. Alternatively, the apparatus 1 may be an independent member and not be included in an apparatus. The sensing unit 11, the calculation unit 12 and/or the determination unit 13 may form a part of or be included in a system, where the system may include a graphical user interface, external hardware devices, a computer processor, a computer network server or other similar signal processing equipment.

Examples of the hemadynamometer include a sphygmomanometer and an automatic blood pressure monitor. Examples of the sphygmomanometer include a stand-type sphygmomanometer, an aneroid-type sphygmomanometer, and a mobile sphygmomanometer. Examples of the automatic blood pressure monitor include upper arm-type automatic blood pressure monitors, wrist-type automatic blood pressure monitors, and finger-type automatic blood pressure monitors, which are classified according to where blood pressure is measured.

The apparatus 1 of the present invention is an apparatus for detecting an optimal site on a human body for measuring blood pressure. Examples of a method of measuring blood pressure include direct/indirect methods, invasive/noninvasive methods, and intrusive/non-intrusive methods. The term blood pressure refers to a pressure on the walls of blood vessels as blood that is pumped out of the heart flows along the blood vessels. In addition, blood pressure includes arterial blood pressure, capillary blood pressure, and venous blood pressure, according to the blood vessel where the blood pressure is measured. The arterial blood pressure varies according to heartbeats. Also, blood pressure includes a systolic pressure when blood flows into the artery as the ventricles of the heart contract, and a diastolic pressure affecting the arterial wall due to the elasticity of the arterial wall even when the ventricles expand and blood stays in the ventricles.

The direct method of measuring blood pressure involves directly inserting a catheter into, for example, the carotid arteries, and connecting the catheter to a manometer to measure blood pressure. The indirect method of measuring blood pressure involves winding a cuff around an upper arm of a human subject, pumping air into the cuff to press on the upper arm, and measuring blood pressure when blood in the brachial artery stops flowing. The invasive method measures blood pressure in the state where a catheter is directly inserted into a blood vessel of a human subject. The noninvasive method measures blood pressure without inserting anything into the blood vessel the human subject. The intrusive method uses a cuff to measure blood pressure. The nonintrusive method does not use a cuff to measure blood pressure.

Where the invasive method includes the direct insertion of the catheter into the blood vessel, blood pressure may be accurately and continuously measured. Examples of the noninvasive method include an auscultatory method of measuring blood pressure using Korotkoff sounds, an oscillometric method of measuring blood pressure using vibrations generated due to the flow of blood, a method using a tonometer, and a method using pulse transit time (“PTT”). Since the auscultatory method and the oscillometric method need expansion or contraction of a cuff, these methods are intrusive and may not continuously measure blood pressure. The method using a tonometer may continuously measure blood pressure. However, the tonometer is a very sensitive instrument. The method using PTT involves using a time interval between a peak of electrocardiography (“ECG”) and a peak of an R-wave of photoplethysmography (“PPG”), has invasive and nonintrusive characteristics, and may continuously measure blood pressure.

A method of measuring blood pressure by pressurizing a particular site of a human body may be used in convenient and portable wrist-type hemadynamometers. For the method of measuring blood pressure by pressurizing a particular site of human body, a sensor included in an apparatus for measuring blood pressure may be located close to a radial artery to acquire accurate data. Also, with respect to the radial artery, if blood pressure is measured at a site corresponding to a portion of the radial artery close to a skin surface, the level of accuracy of blood pressure measurement may be increased.

The apparatus 1 according to the illustrated exemplary embodiment may be applied to all the methods of measuring blood pressure described above. Accordingly, according to exemplary embodiments of the present invention, an optimal blood-pressure measurement site may be detected without use of additional components.

Referring again to FIG. 1, while pressure is applied to a site of a human body at which blood pressure is to be measured, the sensing unit 11 uses at least one sensor and senses a pressure affecting a blood vessel of the pressurized site. In exemplary embodiments, the sensor may be a pressure sensor, but is not limited thereto. In one exemplary embodiment, the sensor may be any apparatus that detects pressure of the blood vessel. The sensing unit 11 may include a plurality of sensors. Where the sensing unit 11 includes the plurality of the sensors, the respective sensors may sense pressures of different sites of a body, or one sensor may sense pressures of different sites of a body while moving the sensors to the different sites.

The apparatus 1 according to the exemplary embodiment may be applied to any site of a body at which blood pressure is to be measured. However, hereinafter, an exemplary embodiment in which a blood-pressure measurement site is determined using a wrist-type hemadynamometer will be exemplarily described.

The sensing unit 11 senses a pressure applied to a blood vessel of a wrist, such as of a human body, that is pressurized to measure blood pressure. An actuator may be used to apply pressure to the blood-pressure measurement site. Pressure is applied to the wrist by using a pressurizing unit (not shown). A pressurizing method may be an entire pressurizing method using a cuff, or a regional pressurizing method of applying pressure to a portion of the blood vessel. However, the apparatus 1 is not limited to these pressurizing methods and may be any type of pressurizing methods to apply pressure to the site for measuring blood pressure.

In one exemplary embodiment, the pressurizing unit applies pressure to the blood-pressure measurement site while gradually increasing the applied pressure, and when the level of the applied pressure reaches an end level, the pressurizing stops. The end level is a pressure value at which the blood flow in an artery stops, and may be variously selected by a user. The sensing unit 11 measures a pressure in the blood vessel of the pressurized site for a time period. That is, the sensing unit 11 measures a pressure in the blood vessel during a time period from before or at the point when the pressurizing unit begins to apply pressure, to the point when or after the pressurizing stops. The time period may be variously selected by a user, and may be from when the flow of arterial blood stops to when the arterial blood normally circulates.

The sensing unit 11 measures a pressure in the blood vessel for a period of time, and transmits the measured values to the calculation unit 12. The sensing unit 11 uses at least one sensor and senses a pressure in the blood vessel of at least one site, and transmits the respective sensed values to the calculation unit 12. In the sensing unit 11, one (single) sensor may sense pressures with respect to a plurality of sites while moving the sensor to determine an optimal blood-pressure measurement site, or an array of a plurality of sensors may simultaneously sense pressures with respect to a plurality of sites to determine an optimal blood-pressure measurement site.

The calculation unit 12 calculates an envelope using the sensed pressures acquired by the sensing unit 11, analyzes the shape of the envelope, and transmits the analysis results to the determination unit 13. The sensing unit 11 uses at least one sensor to measure pressure at least one site, and transmits the acquired sensed pressures to the calculation unit 12. Accordingly, the calculation unit 12 may perform the following exemplary embodiment of a calculating process on data acquired with respect to a plurality of sites.

Hereinafter, an exemplary embodiment of a process of calculating one measurement value acquired with respect to one site will be described. However, those of ordinary skill in the art may easily understand that a plurality of data may also be calculated. FIG. 2 is a detailed block diagram of the calculation unit 12 illustrated in FIG. 1. Referring to FIG. 2, the calculation unit 12 may include a filtering unit 121, an envelope calculation unit 122, a moving average calculation unit 123, a maximum value detection unit 124, and a division unit 125.

The filtering unit 121 allows a high frequency band of sensed pressures from among the sensed pressures acquired by the sensing unit 11 to pass therethrough, and transmits the high frequency band of sensed pressures to the envelope calculation unit 122. The filtering unit 121 may allow a higher frequency band of signals than a boundary frequency to pass therethrough without reduction, and may reduce a cutoff frequency band of signals that are lower than the boundary frequency. The boundary frequency may be determined by combining inductance, a condenser, and resistance.

The sensed pressures acquired by the sensing unit 11 may include an alternating current (“AC”) component and a direct current (“DC”) component. In an exemplary embodiment, to detect a blood-pressure measurement site, the AC component of sensed pressures is used. Thus, the DC component is removed using a high pass filter. The filtering unit 121 is one of high pass filters that are well known to those of ordinary skill in the art. Thus, the filtering unit 121 will not be described in detail herein.

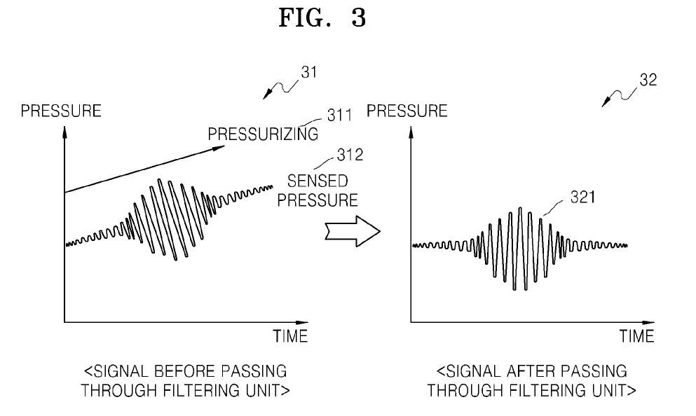

FIG. 3 is a diagram of an exemplary embodiment of a signal process performed by the filtering unit 121. Referring to FIG. 3, a signal 31 indicates a signal before passing through the filtering unit 121, that is, a signal that is transmitted by the sensing unit 11, and a signal 32 indicates a signal after passing through the filtering unit 121. A graph of the signal 31 that has not passed through the filtering unit 121 shows pressure values 311 of pressure applied by the pressurizing unit and sensed pressures 312 of pressure sensed by the sensing unit 11. As described above, the pressure values 311 of pressure applied by the pressurizing unit are increased to a particular level and when they have reached the particular level, the pressurizing stops. The sensed pressures 312 of pressure sensed by the sensing unit 11 include the DC component and the AC component. The filtering unit 121 allows high frequency signals to pass therethrough, and reduces low frequency signals. Accordingly, when sensed pressures 312 of pressure sensed by the sensing unit 11 pass through the filtering unit 121, a waveform 321 including high frequency signals is formed.

Referring to FIG. 2, the envelope calculation unit 122 calculates the envelope of high frequency signals of sensed pressures acquired by the filtering unit 121. To calculate the envelope of high frequency signals acquired by the filtering unit 121, high frequency signals are divided into at least one of point and a maximum value of each point is connected to each other, thereby forming the envelope. In an exemplary embodiment, the maximum value of each point may be calculated by Hilbert transformation.

The moving average calculation unit 123 re-constructs the envelope acquired by the envelope calculation unit 122 using a moving average calculation method. The term moving average refers to an average calculated at different points to identify a change in trend. The average calculation method is a statistic calculation method in which an irregular part of the sensed pressures is removed to find a long-term trend. For the apparatus 1 to more accurately measure blood pressure, the moving average is calculated and the shape of the envelope is analyzed. When the moving average calculation unit 123 calculates the moving average at N points, the moving average may be an N point moving average. For example, if the moving average is measured at three points, the moving average may be referred to as a three point moving average. An exemplary embodiment of the moving average calculation unit 123 according to the present invention may be described with respect to the three point moving average. However, when the moving average is calculated, the number of points is not limited thereto.

In one exemplary embodiment, points that form the envelope acquired by the envelope calculation unit 122 are denoted by a₁, a₂, a₃, through, aₖ, and a₁, a₂, a₃, through, aₖ form a first set of signals A. The signal set A may be defined as in Equation 1.

A={a₁, a₂, a₃, a₄, . . . , aₖ} [Equation 1]

With respect to signals acquired by the filtering unit 121, a₁ denotes a signal value at a first point calculated by the envelope calculation unit 122, and a₂ denotes a signal value at a second point. In this case, k is a natural number and may be set variously.

If B denotes a second set of signals, after the first set of signals A that pass through the envelope calculation unit 122 passes through the moving average calculation unit 123, B may be defined as in Equation 2.

B={b₂, b₃, b₄, b₅, . . . , bₖ₋₁} [Equation 2]

b₂ denotes a value corresponding to a second point signal value calculated by the envelope calculation unit 122, and b₃ denotes a value corresponding to a third point signal value. Members of the second set B may be defined as in Equation 3.

$${b}_{2} = \frac{{a}_{1} + {a}_{2} + {a}_{3}}{3} , {b}_{3} = \frac{{a}_{2} + {a}_{3} + {a}_{4}}{3} , … \qquad Equation 3 ]$$

If the moving average calculation method is generalized, the members of the second set B may be defined as in Equation 4.

$${b}_{x} = \frac{{a}_{x - 1} + {a}_{x} + {a}_{x + 1}}{3} \qquad [ Equation 4 ]$$

If x is a natural number, the three point moving average calculation method is defined as in Equation 4, and bₓ represents a value at an aₓ point. The moving average calculation unit 123 re-constructs the envelope acquired by the envelope calculation unit 122, using Equation 4.

The maximum value detection unit 124 detects a maximum value from among values that form the envelope acquired by the moving average calculation unit 123. If B denotes the second (or subsequent) set of values calculated by the moving average calculation unit 123, the maximum value is detected from among the values that form set B.

The division unit 125 divides the values calculated by the moving average calculation unit 123, by the maximum value detected by the maximum value detection unit 124. In one exemplary embodiment, if B is the subsequent set of values acquired by the moving average calculation unit 123, and bₘ is the maximum value detected by the maximum value detection unit 124, B_{D} that is the set of values acquired by the division unit 125, may be defined as in Equation 5.

$${B}_{D} = \{ \frac{{b}_{2}}{{b}_{m}} , \frac{{b}_{3}}{{b}_{m}} , \frac{{b}_{4}}{{b}_{m}} , … , \frac{{b}_{m - 1}}{{b}_{m}} , 1 , … , \frac{{b}_{m + 1}}{{b}_{m}} , … , \frac{{b}_{k - 1}}{{b}_{m}} \} \qquad [ Equation 5 ]$$

If bₘ is the maximum value from among the values acquired by the moving average calculation unit 123, the maximum value detection unit 124 detects bₘ and the division unit 125 divides the values acquired by the moving average calculation unit 123 by bₘ.

The filtering unit 121, the envelope calculation unit 122, the moving average calculation unit 123, the detection unit 124 and/or the division unit 125 may form a part of or be included in a system, where the system may include a graphical user interface, external hardware devices, a computer processor, a computer network server or other similar signal processing equipment.

Referring to FIG. 1, the determination unit 13 determines, by using the values acquired by the calculation unit 12, whether a site with respect to the values is an optimal blood-pressure measurement site. The determination unit 13 compares the values acquired by the division unit 125 with a reference value and analyzes the shape of a waveform. If the optimal blood-pressure measurement site is determined using only a maximum value of the blood pressure measurement waveform, when the hemadynamometer malfunctions and high pressure is applied and thus a maximum value of sensed pressures is high, or the maximum value has errors due to noise, the blood-pressure measurement site may be wrongly determined. In contrast, as a criterion for determining the blood-pressure measurement site, the shape of the waveform is used instead of the maximum value and thus, the optimal blood-pressure measurement site may be accurately detected.

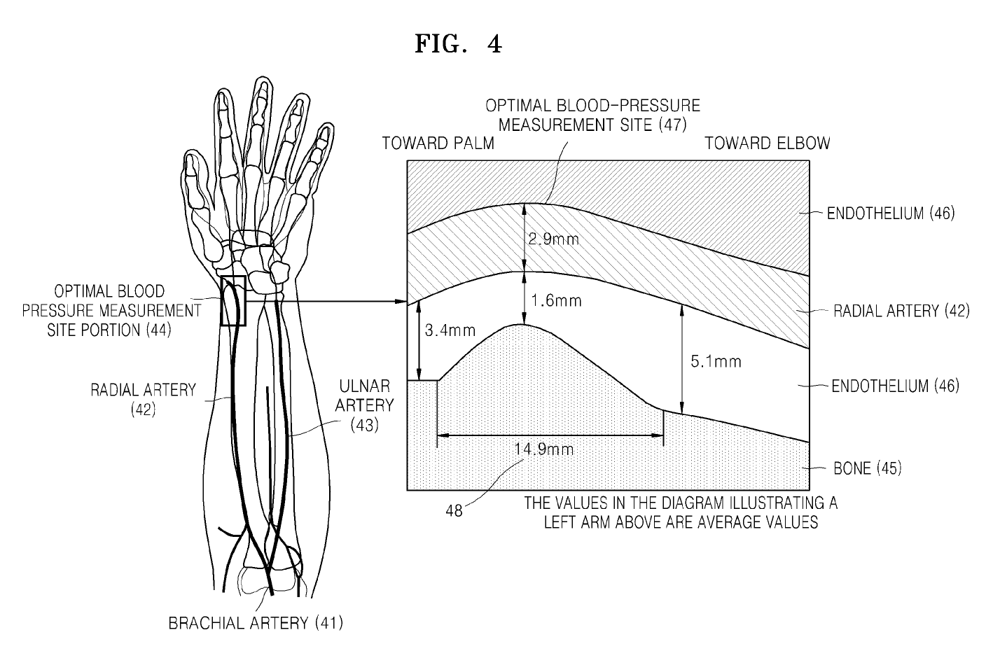

If it is assumed that the apparatus 1 according to the illustrated embodiment is attached to a wrist-type hemadynamometer, the optimal blood pressure measurement site is a site corresponding to a portion of a radial artery closest to a skin surface. FIG. 4 is a diagram of the radial artery located in a wrist, such as of a human arm. Referring to FIG. 4, a brachial artery 41 is divided into a radial artery 42 and an ulnar artery 43. The apparatus 1 according to the illustrated embodiment determines a site corresponding to a portion of a radial artery 42 closest to a skin surface.

A diagram of an exemplary embodiment of a horizontal cross section of an optimal blood-pressure measurement site 44 relative to the wrist, illustrates a bone 45, an endothelium 46 and a radial artery 42. For the radial artery 42, the optimal blood-pressure measurement site 44 corresponds to the portion of the radial artery 42 closest to a skin surface. The optimal blood-pressure measurement site 44 corresponds to the portion of the radial artery 42 that is curved from an inner area of the arm and toward the skin surface. As shown the horizontal cross section of FIG. 4, the radial artery 42 portion is shown curved toward an upward (e.g., skin surface) direction. Since the optimal blood-pressure measurement site 44 corresponds to the portion of the radial artery 42 closest to the skin surface, when pressure applied to the radial artery 42 is measured, the optimal blood-pressure measurement site 44 may be least affected by other sites (for example, endothelium 46).

With reference to the horizontal cross sectional diagram of the optimal blood-pressure measurement site 44 illustrated in FIG. 4, a portion 48 of the radial artery 42 corresponding to the optimal blood-pressure measurement site 44 is located between portions of the endothelia 46 that are thinner (e.g., in a vertical direction of FIG. 4) than other portions of the endothelia 46, and therefore closest to the skin surface. A width of the portion 48 of the radial artery 42 corresponding to the optimal blood-pressure measurement site 44 is illustrated as being about 14.9 millimeters (mm).

FIG. 4 illustrates optimal blood-pressure measurement site 44 disposed overlapping an of the endothelium 46 having a thickness of about 1.6 mm, where adjacent portions of the endothelium 46 increase in thickness, such as to about 3.4 mm and 5.1 mm, respectively. If the uppermost surface of the box in the cross sectional diagram is considered the skin surface (e.g., outer surface of the body), the portion 48 of the radial artery 42 corresponding to the optimal blood-pressure measurement site 44 is closest to this uppermost surface. Thus, at the portion 48 of the radial artery 42, blood pressure may be most accurately measured.

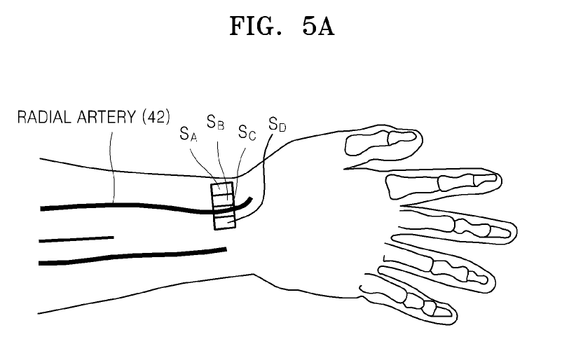

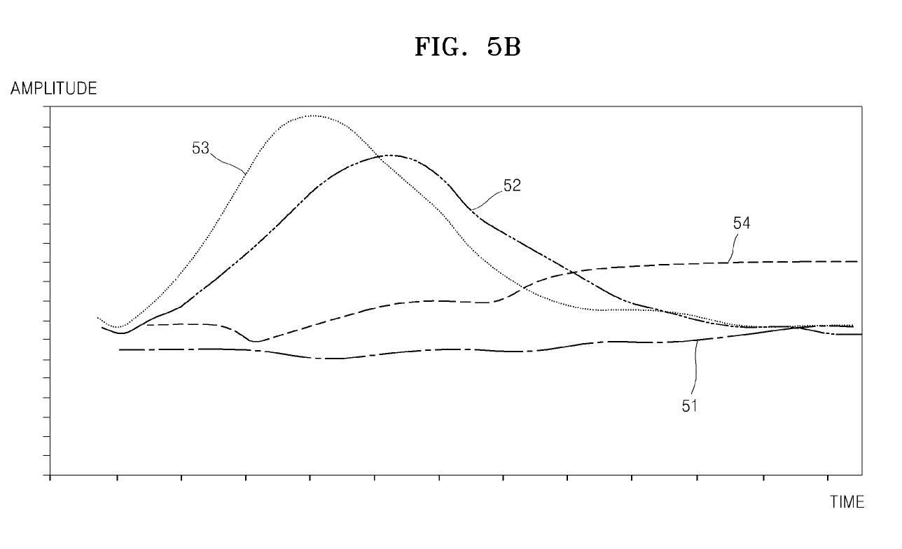

FIG. 5A is a diagram of an exemplary embodiment of a group of sensors arranged close to (e.g., directly adjacent to, adjacent in consecutive sequence or overlapping) a radial artery of a wrist, and FIG. 5B is a diagram of an exemplary embodiment of waveforms of sensed pressures acquired by the respective sensors in FIG. 5A. Referring to FIG. 5A, an array of four identical sensors S_{A}, S_{B}, S_{C}, and S_{D} is disposed close to and substantially perpendicular to a radial artery 42. FIG. 5B is a diagram of sensed pressures acquired by the respective sensors disposed as illustrated in FIG. 5A. The waveforms of 5B are waveforms that have been passed through the filtering unit 121 and the envelope calculation unit 122 of the calculation unit 12.

Referring to FIG. 5B, a graph 51 shows sensed pressures acquired by the sensor S_{A}, a graph 52 shows sensed pressures acquired by the sensor S_{B}, a graph 53 shows sensed pressures acquired by the sensor S_{C}, and a graph 54 shows sensed pressures acquired by the sensor S_{D}. Referring to FIG. 5A, the sensor S_{C} is disposed on the skin surface located vertically above (e.g. overlapping) the radial artery 42. Referring to FIG. 5B, the maximum value of the graph 53 illustrating sensed pressures acquired by the sensor S_{C} is largest, and also the acquired sensed pressures of the graph 53 most rapidly decrease.

Referring to FIG. 5B, a waveform represented by the graph 53 of sensed pressures measured at the site vertically above the radial artery 42 has a bell shape having the narrowest width. Since the sensor S_{A} and the sensor S_{D} are located relatively far from the site located vertically above the arterial blood vessel, the graph 51 illustrating sensed pressures acquired by the sensor S_{A} and the graph 54 illustrating sensed pressures acquired by the sensor S_{D} have small maximum values and large widths in comparison to the graph 53.

The graph 51 illustrating sensed pressures acquired by the sensor S_{A} and the graph 54 illustrating sensed pressures acquired by the sensor S_{D} have small maximum values and large widths in comparison to the graph 53 because when a sensor measures blood pressure applied to the wall of a blood vessel, the blood pressure may not be accurately measured due to resistance of other sites. That is, when a waveform has a maximum value, sensed pressures acquired are sequentially decreased over time based on a time axis at which the maximum value is detected, and acquired sensed pressures are reduced equal to or less than a reference value that is smaller than the maximum value, the site of a body corresponding to the waveform is determined as an optimal blood-pressure measurement site.

The reference value may be a value input by a user, a value that is stored in advance as a default value in the determination unit 13, or a value that is acquired by an external apparatus. The external apparatus may be any other apparatus that is connected to the apparatus 1. In an exemplary embodiment, the reference value may be input by a user using a keyboard, or a value that is input as a default value. Since the reference value is used to determine whether the waveform has the bell shape, the reference value may be less than the maximum value. In one exemplary embodiment, the reference value may be set variously, and may be a value corresponding to 50% of the maximum value, or a value corresponding to 30% of the maximum value.

Accordingly, the graph 52 illustrating sensed pressures acquired by the sensor S_{B} and the graph 53 illustrating sensed pressures acquired by the sensor S_{C}, which are waveforms acquired by the sensor S_{B} and sensor S_{C} that sense blood pressure at a site close to radial artery 42, have the most bell-like shapes. Among the two waveforms 52 and 53, the waveform having the narrow width, that is, the graph 53 illustrating sensed pressures acquired by the sensor S_{C}, is the most appropriate for measuring blood pressure. Accordingly, the site at which blood pressure is measured by the sensor S_{C}, that is, a portion of the skin surface disposed vertically above (e.g., directly overlapping) the radial artery 42, may be determined as the blood-pressure measurement site providing high accuracy.

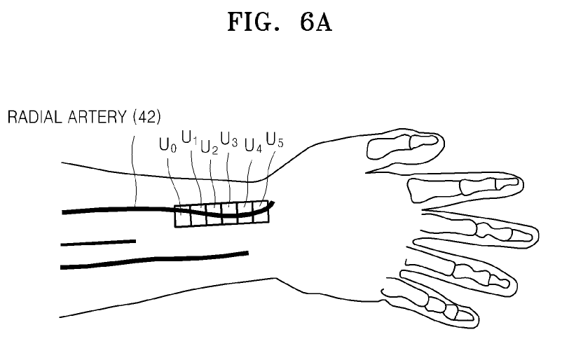

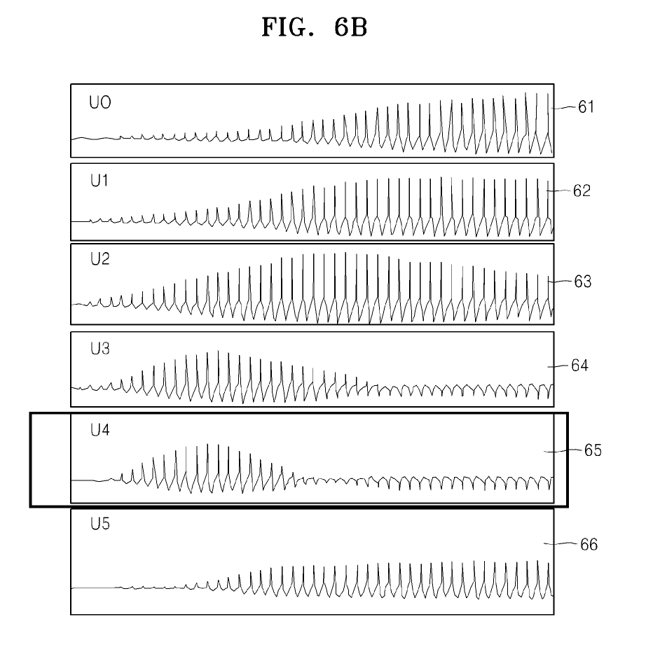

Referring to FIG. 4, it may be identified that, for the radial artery 42, the site 47 of the radial artery 42 that slightly protrudes toward the skin surface is most appropriate for measuring the blood pressure. FIG. 6A is a diagram of an exemplary embodiment of a group of sensors longitudinally arranged along a longitudinal extension direction of a radial artery 42 of a wrist, and FIG. 6B is a diagram of an exemplary embodiment of waveforms of sensed pressures acquired by the respective sensors in FIG. 6A.

Referring to FIG. 6A, each of identical sensors U₀, U₁, U₂, U₃, U₄, and U₅ are disposed on a portion of the skin surface vertically above (e.g., overlapping) the radial artery 42, and waveforms acquired by the respective sensors are illustrated in FIG. 6B. As described with reference to FIG. 5B, FIG. 6B illustrates graphs of waveforms that pass through the filtering unit 121 of the calculation unit 12.

Referring to FIG. 6B, it may be identified that even when blood pressure is measured at the site of the skin surface disposed vertically above the radial artery 42 and along the longitudinal direction of the radial artery 42, the acquired waveforms are different. That is, even along the longitudinal direction of the radial artery 42, there is an optimal site for measuring blood pressure. A graph 61 shows sensed pressures acquired by the sensor U₀, a graph 62 shows sensed pressures acquired by the sensor U₁, a graph 63 shows sensed pressures acquired by the sensor U₂, a graph 64 shows sensed pressures acquired by the sensor U₃, a graph 65 shows sensed pressures acquired by the sensor U₄, and a graph 66 shows sensed pressures acquired by the sensor U₅.

Referring to FIG. 6B, the waveforms acquired by the sensor U₃ and U₄ have a profile closest to bell shapes. That is, a waveform 65 acquired by the sensor U₄ located in the optimal blood-pressure measurement site of the radial artery 42, for example, at a site of the skin surface closest to the radial artery 42, and a waveform 64 acquired by the sensor U₃ located close to the sensor U₄ have bell shapes. As described above, when a waveform has the bell shape, a bell-shaped waveform having a large absolute maximum value and a high decrease rate (e.g., slope) from the maximum value over time, is a waveform acquired with respect to a site where blood pressure measurement is highly accurate. Accordingly, it may be identified by referring to FIG. 6B that the waveform 65 acquired by the sensor U₄ is a waveform from a site that is most appropriate for measuring blood pressure, and the sensor U₄ is located at a site of the skin surface closest to the radial artery 42.

As described above, regarding methods of determining a blood-pressure measurement site, when a method of determining a site at which a waveform having the largest maximum value is used, a maximum value acquired by continuously applying pressure and a maximum value acquired using different amounts and/or degrees of pressure applied and pressurizing methods are detected, or a signal generated by noise is detected as a maximum value. Thus, it is highly likely that the blood-pressure measurement site may be wrongly determined. Accordingly, the determination unit 13 according to the illustrated embodiment of the present invention analyzes the shape of a waveform, instead of the maximum value, to determine the blood-pressure measurement site.

Referring to FIG. 1, the determination unit 13 acquires values acquired by the moving average calculation unit 123, which are divided by the maximum value by the division unit 125. The determination unit 13 compares the acquired values with a reference value, and determines whether the blood-pressure measurement site is an appropriate site based on whether there are values equal to or less than the reference value.

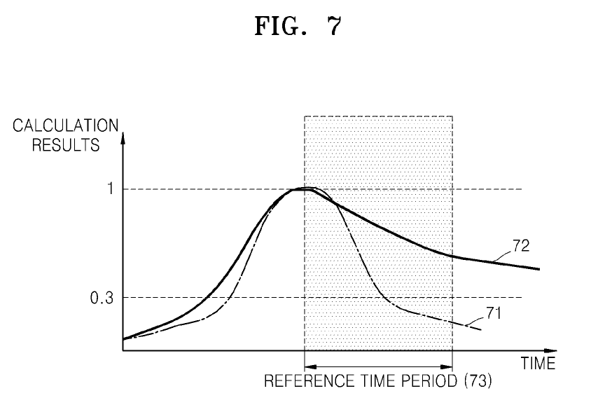

FIG. 7 is a diagram illustrating an exemplary embodiment of envelopes of sensed pressures in order to determine a blood-pressure measurement site. Referring to FIG. 7, a graph 71 shows values with respect to an optimal blood-pressure measurement site acquired by the calculation unit 12, and a graph 72 shows values with respect to a site that is located vertically above (e.g., overlapping) the radial artery 42 and outside the optimal blood-pressure measurement site acquired by the calculation unit 12. An absolute maximum value of the graph 71 is referred to as a first maximum value, and an absolute maximum value of the graph 72 is referred to as a second maximum value.

In the illustrated embodiment, if the reference value is set to 0.3, the graph 71 illustrating the values with respect to the optimal blood-pressure measurement site has values equal to or less than 0.3 after the first maximum value. On the other hand, the graph 72 showing the values with respect to the site close to the radial artery 42, but outside of the optimal blood-pressure measurement site, does not have values equal to or less than 0.3 after the second maximum value. Accordingly, the determination unit 13 compares the reference value with values acquired by the division unit 125 and determines a site corresponding to a waveform that has a value equal to or less than the reference value as the optimal blood-pressure measurement site. However, the reference value is not limited to 0.3. In one exemplary embodiment, the reference value may be more than 0 and less than 1 and also, may be any reference value that is used to determine whether the shape of a waveform is the bell shape.

Also, when some of the values acquired by the division unit 125 are equal to or less than the reference value within a reference time period, the determination unit 13 may determine a site corresponding to a waveform of the values acquired by the calculation unit 12 as the optimal blood-pressure measurement site. When the waveform of the values acquired by the calculation unit 12 is too wide and some values are equal to or less than the reference value after a long time period (e.g., past the reference time period), a site at which blood pressure is measured may be wrongly determined as an appropriate site. Accordingly, a time may be set to a reference time period 73 (FIG. 7), and only a waveform having values equal to or less than the reference value within that reference time period 73 may be determined as an appropriate waveform.

As described above, when a plurality of sites are sensed and a plurality of waveforms are used to determine an optimal blood-pressure measurement site, a site corresponding to a waveform that is first to have a value equal to or less than the reference value after a maximum value, from among the waveforms may be determined as the optimal blood-pressure measurement site.

Also, when any value acquired by the division unit 125 is greater than the reference value, and a time corresponding to the maximum value after blood pressure begins to be measured is outside a threshold time, the determination unit 13 may determine that a blood-pressure measurement site is outside the radial artery, or that the blood-pressure measurement site has an error. In this case, the threshold time period may vary according to environments used and the type of hemadynamometer used.

In one exemplary embodiment, the threshold time may be about 10 seconds or about 20 seconds. If the threshold time is 10 seconds, when the maximum value is not detected for 10 seconds after the blood pressure begins to be measured, the determination unit 13 determines that the blood-pressure measurement site is not the optimal blood-pressure measurement site. Referring to FIG. 5B, the waveform 54 acquired by the sensor S_{D} does not have the bell shape, and pressure applied to a blood vessel is continuously increased. That is, if the maximum value is measured long after (e.g., exceeding a time period) the blood pressure begins to be measured, the blood-pressure measurement site is outside the radial artery 42, or a blood pressure value is high due to the continuous application of pressure, or errors may occur due to noise. Accordingly, by using the threshold time for detecting the maximum value, the determination unit 13 may remove factors hindering accurate blood pressure measurement.

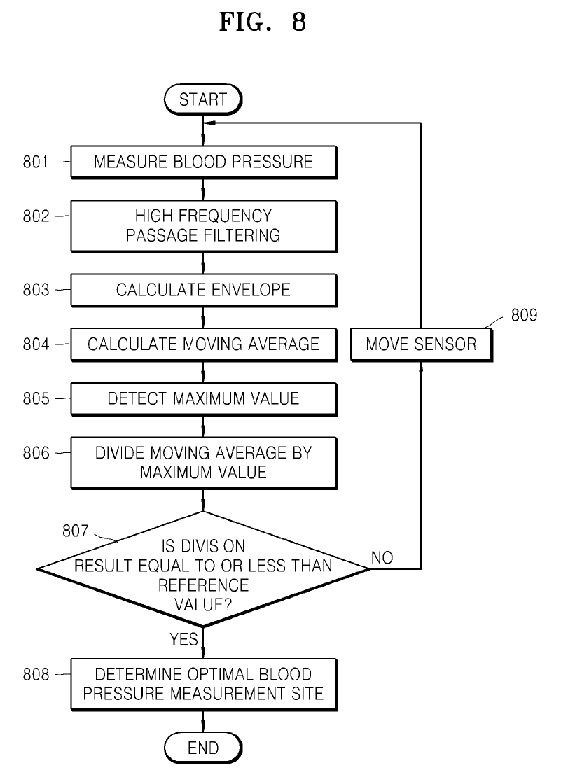

FIG. 8 is a flowchart illustrating an exemplary embodiment of a method of detecting a blood-pressure measurement site by using one sensor according to the present invention. Referring to FIG. 8, the method of detecting a blood-pressure measurement site according to the illustrated embodiment includes sequential operations performed by the apparatus illustrated in FIG. 1. Thus, even though not described hereinafter, the description of the apparatus illustrated in FIG. 1 that has been presented is also effective in the method of detecting a blood-pressure measurement site according to the illustrated embodiment.

The sensing unit 11 includes at least one sensor, and FIG. 8 is a flowchart illustrating a method of detecting a blood-pressure measurement site by using only one sensor while moving the only one sensor.

In operation 801, the sensing unit 11 measures blood pressure. That is, the sensing unit 11 measures blood pressure applied to a blood vessel while applying pressure to a blood-pressure measurement site. Alternatively, when blood pressure is measured using direct methods, blood pressure in a blood vessel is measured without applying pressure.

In operation 802, the sensed pressures acquired by the sensing unit 11 are filtered by the filtering unit 121 so that high frequency values remain. Once the values acquired by the sensing unit 11 are passed through the filtering unit 121, high frequency values of the acquired values remain.

In operation 803, an envelope of values acquired by the filtering unit 121 is calculated by the calculation unit 122.

In operation 804, a moving average of values acquired by the envelope calculation unit 122 is calculated by the moving average calculation unit 123. The moving average calculation unit 123 calculates a moving average at points, such as determined by a user, and re-constructs the envelope.

In operation 805, a maximum value is detected among the moving averages acquired by the moving average calculation unit 123.

In operation 806, the values acquired by the moving average calculation unit 123 are divided by the maximum value acquired by the maximum value detection unit 124.

In operation 807, the division results acquired by the division unit 125 are compared with a reference value by the determination unit 13. If a value that is equal to or less than the reference value is not present, operation 809 is performed. If a value that is equal to or less than the reference value is present, operation 808 is performed.

In operation 808, if the value that is equal to or less than the reference value is present, a site at which the sensor measures is determined as an appropriate blood-pressure measurement site.

In operation 809, if the value that is equal to or less than the reference value is not present, the site at which the sensor measures is determined as an inappropriate blood-pressure measurement site. Thus, the sensor is moved (e.g., physically on the body) and the operations 801 through 807 are performed again.

A portion of or an entire of operations 801 to 808 may be performed in a processing system, which may include a computer processor, computer network server and/or other signal processing equipment. Results of measuring the blood pressure in operation 801, filtering the sensed pressures acquired by the sensing unit 11 in operation 802, calculating the envelope of values acquired by the filtering unit 121 in operation 803, calculating a moving average of values acquired by the envelope calculation unit 122 in operation 804, detecting a maximum value among the moving averages acquired by the moving average calculation unit 123 in operation 805, dividing the values acquired by the moving average calculation unit 123 by the maximum value acquired by the maximum value detection unit 124 in operation 806, comparing the division results acquired by the division unit 125 with a reference value by the determination unit 13 in operation 807, and/or determining an optimal blood pressure measurement site if the value that is equal to or less than the reference value is present at operation 808 may be outputted to a user, such as via a graphical user interface, or to an external (e.g., hardware) device such as a printer, a computer processor, computer network server or other signal processing equipment.

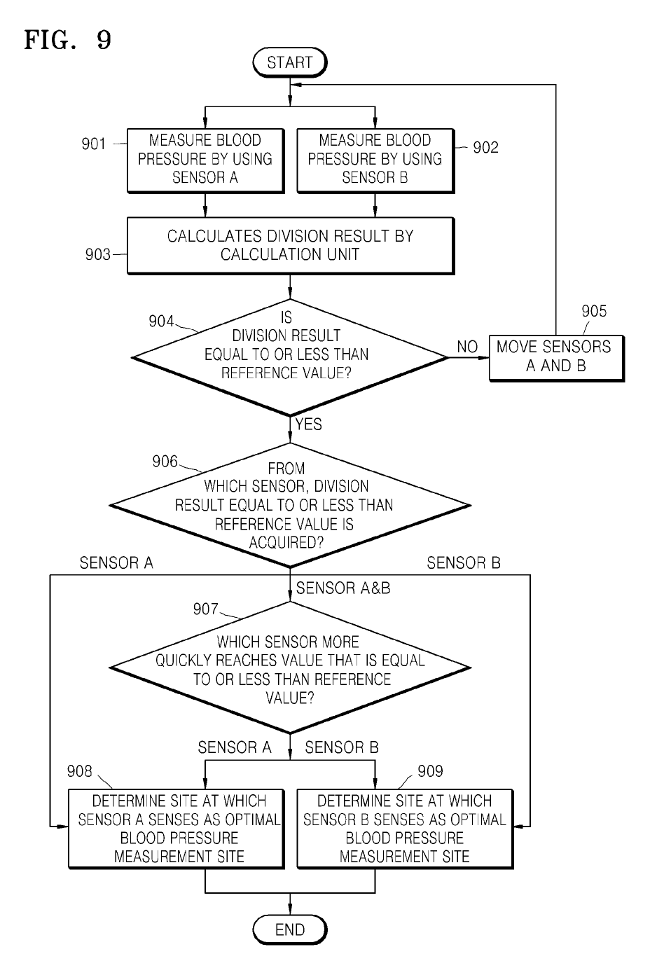

FIG. 9 is a flowchart illustrating exemplary embodiment of a method of detecting a blood-pressure measurement site by using two sensors according to the present invention. Referring to FIG. 9, the method of detecting a blood-pressure measurement site according to the illustrated embodiment includes sequential operations performed by the apparatus illustrated in FIG. 1. Thus, even though not described hereinafter, the description of the apparatus illustrated in FIG. 1 that has been presented is also effective in the method of detecting a blood-pressure measurement site according to the illustrated embodiment.

The sensing unit 11 according to the illustrated embodiment includes at least one sensor. FIG. 9 is a flowchart illustrating a method of detecting an optimal blood-pressure measurement site by using a plurality of individual sensors. Although the method illustrated in FIG. 9 uses two exemplarily sensors A and B, the number of sensors used is not limited thereto and a plurality of sensors greater than two may also be used to detect the optimal blood-pressure measurement site.

In operations 901 and 902, the sensing unit 11 uses the sensor A and the sensor B, and each of the sensors A and B measures blood pressure. That is, each of the sensors A and B measures pressure in a blood vessel at first sites in which the respective sensors A and B are located.

In operation 903, the sensed pressures acquired by the sensors A and B are calculated by the calculation unit 12. The operation 903 includes the operations 802 through 806 illustrated in FIG. 8. The calculation may be separately performed on the sensed pressures acquired by the sensors A and B.

In operation 904, it is determined whether the calculation results with respect to the sensors A and B include a value that is equal to or less than the reference value. If the calculation results with respect to at least one of the sensors A and B include a value that is equal to or less than the reference value, operation 906 is performed. Alternatively, if the calculation results with respect to the sensor A and the calculation results with respect to sensor B are all greater than the reference value, operation 905 is performed.

In operation 905, if the calculation results with respect to the sensor A and the calculation results with respect to sensor B are all greater than the reference value, the sensor A and the sensor B are physically moved to alternative measurement sites different from the first measurement sites, and the operations 901 and 902 are performed again.

In operation 906, if the calculation results include the value that is equal to or less than the reference value, the sensor, which has the value that is equal to or less than the reference value, is determined. If the calculation results with respect to the sensor A include the value that is equal to or less than the reference value, operation 908 is performed. If the calculation results with respect to the sensor B include the value that is equal to or less than the reference value, operation 909 is performed. If the calculation results with respect to both the sensor A and the sensor B include the value that is equal to or less than the reference value, operation 907 is performed.

In operation 907, If the calculation results with respect to both the sensor A and the sensor B include the value that is equal to or less than the reference value, it is determined that calculation results with respect to which sensor first includes the value that is equal to or less than the reference value. That is, as a waveform of the calculation results acquired by the calculation unit 12 has a narrower bell shape, the waveform is more appropriate for acquiring a high-accuracy blood pressure value. Thus, a site at which a sensor first includes the value that is equal to or less than the reference value (e.g., from a maximum value) measures is the optimal blood-pressure measurement site. Accordingly, if the values acquired by the sensor A are first to become equal to or less than the reference value, operation 908 is preformed, and if the values acquired by the sensor B are first to become equal to or less than the reference value, operation 909 is performed.

In operation 908, it is determined that a site at which the sensor A measures is the optimal blood-pressure measurement site.

In operation 909, it is determined that a site at which the sensor B measures is the optimal blood-pressure measurement site.

A portion of or an entire of operations 901 to 904 and 906 to 909 may be performed in a processing system, which may include a computer processor, computer network server and/or other signal processing equipment. Results of measuring the blood pressure in operations 901 and 902, calculating a division result of operation 903, determining whether the calculation results with respect to the sensors A and B include a value that is equal to or less than the reference value of operation 904, determining the sensor of the plurality of sensor which has the value that is equal to or less than the reference value of operation 906, determining calculation results with respect to which sensor first includes the value that is equal to or less than the reference value of operation 907, and determining the optimal blood pressure measurement site of operations 908 and 909 may be outputted to a user, such as via a graphical user interface, or to an external (e.g., hardware) device such as a printer, a computer processor, computer network server or other signal processing equipment.

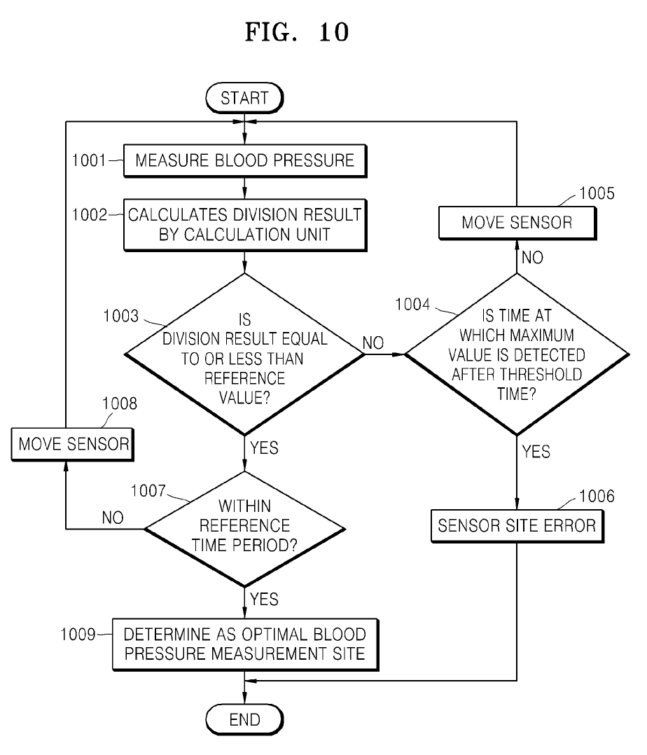

FIG. 10 is a flowchart illustrating exemplary embodiment of a method of detecting a blood-pressure measurement site in which time is taken into consideration, according to the present invention. Referring to FIG. 10, the method of detecting a blood-pressure measurement site according to the illustrated embodiment includes sequential operations performed by the apparatus illustrated in FIG. 1. Thus, even though not described hereinafter, the description of the apparatus illustrated in FIG. 1 that has been presented is also effective in the method of detecting a blood-pressure measurement site according to the illustrated embodiment.

In operation 1001, the sensing unit 11 measures blood pressure.

In operation 1002, the calculation unit 12 calculates division results using measurement results acquired by the sensing unit 11. That is, the operation 1002 includes the operations 802 through 806 illustrated in FIG. 8.

In operation 1003, the determination unit 13 determines whether the division results include a value that is equal to or less than the reference value. If the value that is equal to or less than the reference value is present, operation 1007 is performed. If the value that is equal to or less than the reference value is not present, operation 1004 is performed.

In operation 1004, if the division results do not include the value that is equal to or less than the reference value, the determination unit 13 determines whether the maximum value detected by the maximum value detection unit 124 appears after the threshold time after the blood pressure begins to be measured. In one exemplary embodiment, if the threshold time is 10 seconds, the determination unit 13 determines whether the maximum value appears after 10 seconds after the blood pressure begins to be measured. If the maximum value appears after the threshold time, operation 1006 is performed. If the maximum value appears before or at the threshold time, operation 1005 is performed.

In operation 1005, if the division results do not include the value that is equal to or less than the reference value, and the maximum value appears within a reference time period, for example, within 10 seconds, a site at which a sensor measures is determined as being not far from the radial artery, and the sensor is physically moved to an alternative measurement site which is closer to the radial artery. Once the sensor has moved, the operation 1001 is performed again.

In operation 1006, if the division results do not include the value that is equal to or less than the reference value, and the maximum value appears after a reference time period, for example, after 10 seconds, the site at which the sensor measures is determined as being very far from the radial artery (e.g., further from the radial artery 42 than in operation 1005) and the measurement site is determined as being inappropriate. According to environments used, it may be outputted and/or reported to a user or processing system, that the measurement site is inappropriate, as being ineffectively too far from the radial artery 42. Accordingly, a user could determine or the processing system could indicate that the site at which the sensor at operation 1006 measures is not an optimal site to measure blood-pressure, so that the user could change the location of the sensor and measure the blood-pressure again.

In operation 1007, if the division results include the value that is equal to or less than the reference value, it is determined whether the value that is equal to or less than the reference value appears after the maximum value within a reference time period. If the division results are equal to or less than the reference value and appear within the reference time period, operation 1009 is performed. If the division results become equal to or less than the reference value and appear after the reference time period, operation 1008 is performed.

In operation 1008, if the division results are equal to or less than the reference value after the reference time period, a site at which the sensor measures is determined as a site of the radial artery that is not closest to the skin surface and the sensor is physically moved to an alternative measurement site. Once the sensor is moved, the operation 1001 is performed again.

In operation 1009, if the division results are equal to or less than the reference value before the reference time period expires, the site at which the sensor measures is determined as an appropriate and optimal blood-pressure measurement site. The determination result may be reported to a user, blood pressure may be measured at the site, or blood pressure that is actually measured may be calculated using measurement values with respect to the site.

A portion of or an entire of operations 1001 to 1004, 1007 and 1009 may be performed in a processing system, which may include a computer processor, computer network server and/or other signal processing equipment. Results of measuring the blood pressure in operation 1001, calculating a division result of operation 1002, determining whether the division results include a value that is equal to or less than the reference value of operation 1003, determining whether the maximum value detected by the maximum value detection unit 124 appears after the threshold time after the blood pressure begins to be measured of operation 1004, is determining whether the value that is equal to or less than the reference value appears after the maximum value within a reference time period of operation 1007, and determining an appropriate and optimal blood-pressure measurement site of operation 1009 may be outputted to a user, such as via a graphical user interface, or to an external (e.g., hardware) device such as a printer, a computer processor, computer network server or other signal processing equipment.

Accordingly, the optimal blood-pressure measurement site may be easily detected, and blood pressure may be measured at the detected optimal blood-pressure measurement site, thereby improving reliability of blood pressure measurement. When a method of measuring blood pressure by applying pressure to a particular site is used, blood pressure may be continuously and accurately measured.

As described above, according to the one or more of the above embodiments, an optimal blood-pressure measurement site may be easily detected without having to use any additional devices. Also, the reliability of blood pressure measurement results may be improved, and when used in a method of measuring blood pressure by applying pressure to a particular site, blood pressure may be continuously and accurately measured.

In addition, other exemplary embodiments may also be implemented through computer readable code, computer readable instructions which are in and/or on a medium, e.g., a computer readable medium, to control at least one processing element to implement any above described embodiment. The medium may correspond to any medium/media permitting the storage and/or transmission of the computer readable code. The storage and/or transmission of the computer readable code may include the use of a system including a graphical user interface, external hardware devices, a computer processor, a computer network server or other similar signal processing equipment.

The computer readable code may be recorded and/or transferred on a medium in a variety of ways. The medium includes, but is not limited to, recording media, such as magnetic storage media (e.g., ROM, floppy disks, hard disks, etc.) and optimal recording media (e.g., CD-ROMs, or DVDs) and transmission media, as well as elements of the Internet. Thus, the medium may be such a defined and measurable structure including or carrying a signal or information, such as a device carrying a bitstream according to one or more embodiments. The media may also be a distributed network, so that the computer readable code is stored and/or transferred and executed in a distributed fashion. Furthermore, the processing element could include a processor or a computer processor, and processing elements may be distributed and/or included in a single device.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. An apparatus which detects an optimal site of a body to measure blood pressure, the apparatus comprising:
a sensing unit which senses pressures applied to a blood vessel of a site of the body;
a calculation unit which calculates a waveform representing the sensed pressures; and
a determination unit which determines whether the site is the optimal site based on a shape of the waveform.

2. The apparatus of claim 1, wherein the determination unit determines whether the site is the optimal site, based on an increase and a decrease trend of the sensed pressures which are represented by the shape of the waveform.

3. The apparatus of claim 1, wherein when the sensed pressures are continuously decreased as time elapses after a time corresponding to a maximum value from among the sensed pressures which form the waveform, and the sensed pressures comprise values equal to or less than a reference value so that the shape of the waveform is a bell shape, the determination unit determines that the site of the body is the optimal site.

4. The apparatus of claim 3, wherein the reference value is a value which is input by a user, a value which is stored in the determination unit, or a value which is acquired by an external apparatus.

5. The apparatus of claim 1, wherein
the calculation unit calculates ratios of the sensed pressures to a maximum value from among the sensed pressures, and
when calculation results with respect to values after the maximum value are equal to or less than a reference value, the determination unit determines that the site is the optimal site,
wherein the reference value is for determining a decrease rate of the sensed pressures as time elapses after a time at which the maximum value of the waveform is detected.

6. The apparatus of claim 1, wherein the calculation unit comprises:
a filtering unit which filters the sensed pressures;
an envelope calculation unit which calculates an envelope of the filtered values; and
a division unit which divides the values which form the envelope by a maximum value from among the values which form the envelope,
wherein when division results with respect to values after the maximum value are equal to or less than a reference value, the determination unit determines that the site is the optimal site,
wherein the reference value is for determining a decrease rate of the sensed pressures as time elapses after a time at which the maximum value of the waveform is detected.

7. The apparatus of claim 5, wherein when the calculation results with respect to values that appear after a time corresponding to the maximum value are equal to or less than the reference value within a reference time period after the maximum value is detected, the determination unit determines that the site is the optimal site,
wherein the reference time period is input by a user, or stored in the determination unit, or acquired by an external apparatus.

8. The apparatus of claim 1, wherein
the sensing unit senses a plurality of sites of the body by using at least one sensor,
the calculation unit calculates envelopes with respect to the respective sites sensed by the sensor, and
the determination unit determines that a site corresponding to an envelope from among the calculated envelopes having a highest decrease rate of the sensed pressures as time elapses after a time at which a maximum value from among the sensed pressures which form the waveform, is the optimal site.

9. The apparatus of claim 8, wherein in the sensing unit, one sensor senses the plurality of sites of the body at time intervals.

10. The apparatus of claim 8, wherein the calculation unit comprises:
a filtering unit which filters the sensed pressures;
an envelope calculation unit which calculates envelopes of the filtered values with respect to the respective sites of the body; and
with respect to each of the envelopes, a division unit which divides the values which form the envelope by a maximum value from among the values that form the envelope,
wherein the determination unit determines that a site corresponding to an envelope of which division results with respect to values after the maximum value are equal to or less than a reference value, is the optimal site,
wherein the reference value is for determining a decrease rate of the sensed pressures as time elapses after a time at which the maximum value of the waveform is detected.

11. The apparatus of claim 10, wherein when a plurality of envelopes comprise values which are equal to or less than the reference value, the determination unit determines that a site corresponding to an envelope that comprises values that are equal to or less than the reference value for the shortest time period, is the optimal site.

12. The apparatus of claim 1, wherein the body is a human body, and the optimal site is a portion of a radial artery of a wrist that is closest to a skin surface.

13. The apparatus of claim 12, wherein the calculation unit comprises:
a filtering unit which filters the sensed pressures;
an envelope calculation unit which calculates an envelope of the filtered values; and
a division unit which divides the values which form the envelope by a maximum value from among the values which form the envelope,
wherein when the division results with respect to values that are acquired after a time at which the maximum value is detected are greater than a reference value, and the time at which the maximum value is detected is outside a reference time period from when the site of the body begins to be sensed to a reference time, the determination unit determines that the site of the body is outside the radial artery,
wherein the reference value is for determining a decrease rate of the sensed pressures as time elapses after a time at which the maximum value of the waveform is detected.

14. A method of detecting a site of a body to measure blood pressure, the method comprising:
sensing pressures applied to a blood vessel of a site of the body;
calculating a waveform representing the sensed pressures; and
determining whether the site is an optimal site based on a shape of the waveform.

15. The method of claim 14, wherein the determining whether the site is an optimal site is based on an increase and a decrease trend of the sensed pressures which are represented by the shape of the waveform.

16. The method of claim 14, wherein in the determining whether the site is an optimal site, when the sensed pressures are continuously decreased as time elapses after a time corresponding to a maximum value from among the sensed pressures which form the waveform, and the sensed pressures comprise values equal to or less than a reference value so that the shape of the waveform is a bell shape, the site is determined as the optimal site.

17. The method of claim 16, wherein the reference value is a value which is input by a user, a value which is stored in a determination unit, or a value which is acquired by an external apparatus.

18. The method of claim 14, wherein
in the calculating a waveform representing the sensed pressures, ratios of the sensed pressures to a maximum value from among the sensed pressures are calculated, and
in the determining whether the site is an optimal site, when calculation results with respect to values after the maximum value are equal to or less than a reference value, the site is determined as the optimal site,
wherein the reference value is for determining a decrease rate of the sensed pressures as time elapses after a time at which the maximum value of the waveform is detected.

19. The method of claim 14, wherein the calculating a waveform representing the sensed pressures further comprises:
filtering the sensed pressures;
calculating an envelope of the filtered values; and
dividing the values which form the envelope by a maximum value from among the values which form the envelope,
wherein when division results with respect to values after the maximum value are equal to or less than a reference value, the site is determined as the optimal site,
wherein the reference value is for determining a decrease rate of the sensed pressures as time elapses after a time at which the maximum value of the waveform is detected.

20. The method of claim 18, wherein in the determining whether the site is an optimal site, when the calculation results with respect to values which appear after a time corresponding to the maximum value are equal to or less than the reference value within a reference time period after the maximum value is detected, the site is determined as the optimal site,
wherein the reference time period is input by a user, or stored in the determination unit, or acquired by an external apparatus.

21. The method of claim 14, wherein
in the sensing pressures applied to a blood vessel of a site of the body, a plurality of sites of the body is sensed by using at least one sensor,
in the calculating a waveform representing the sensed pressures, envelopes are calculated with respect to the respective sites sensed by the at least one sensor, and
in the determining whether the site is an optimal site, a site corresponding to an envelope from among the calculated envelopes having the highest decrease rate of the sensed pressures as time elapses after a time at which a maximum value from among the sensed pressures that form the waveform is detected, is determined as the optimal site.

22. The method of claim 21, wherein in the sensing pressures applied to a blood vessel of a site of the body, one sensor senses the plurality of sites of the body at time intervals.

23. The method of claim 21, wherein the calculating a waveform representing the sensed pressures comprises:
filtering the sensed pressures;
calculating envelopes of the filtered values with respect to the respective sites of the body; and
with respect to each of the envelopes, the values which form the envelope are divided by a maximum value from among the values which form the envelope,
wherein in the determining whether the site is an optimal site, a site corresponding to an envelope of which division results with respect to values after the maximum value are equal to or less than a reference value, is determined as the optimal site,
wherein the reference value is for determining a decrease rate of the sensed pressures as time elapses after a time at which the maximum value of the waveform is detected.

24. The method of claim 23, wherein in the determining whether the site is an optimal site, when a plurality of envelopes comprise values which are equal to or less than the reference value, a site corresponding to an envelope which comprises values that are equal to or less than the reference value for the shortest time period is determined as the optimal site.

25. The method of claim 14, wherein the body is a human body and the optimal site is a portion of a radial artery of a wrist that is closest to a skin surface.

26. The method of claim 25, wherein the calculating a waveform representing the sensed pressures comprises:
filtering the sensed pressures;
calculating an envelope of the filtered values; and
dividing the values which form the envelope by a maximum value from among the values that form the envelope,
wherein when division results with respect to values that are acquired after a time at which the maximum value is detected are greater than a reference value, and the time at which the maximum value is detected is outside a reference time period from when the site of the body begins to be sensed, it is determined that the site of the body is outside the radial artery,
wherein the reference value is for determining a decrease rate of the sensed pressures as time elapses after a time at which the maximum value of the waveform is detected.

27. A computer readable recording medium storing instructions which control at least one processor to perform a method of detecting a site of a body to measure blood pressure, the method comprising:
sensing pressures applied to a blood vessel of a site of the body;
calculating a waveform representing the sensed pressures; and
determining whether the site is an optimal site based on a shape of the waveform.
